(19) United States
(12) Patent Application Publication
(10) Pub. No.: US 2017/0057888 A1
(43) Pub. Date: Mar. 2, 2017

(54) SEPARATION OF CATALYST AND HYDROGEN ACCEPTOR AFTER AROMATIZATION OF A METHANE CONTAINING GAS STREAM

(71) Applicant: SHELL OIL COMPANY, Houston, TX (US)

(72) Inventors: Ye-Mon CHEN, Sugar Land, TX (US); Richard Addison SANBORN, Estes Park, CO (US); Surya B Reddy KARRI, Naperville, IL (US); Yeook ARRINGTON, Downers Grove, IL (US); John G. FINDLAY, Homer Glen, IL (US)

(21) Appl. No.: 15/245,867

(22) Filed: Aug. 24, 2016

Related U.S. Application Data
(60) Provisional application No. 62/210,648, filed on Aug. 27, 2015.
(60) Provisional application No. 62/257,424, filed on Nov. 19, 2015.
(60) Provisional application No. 62/257,460, filed on Nov. 19, 2015.

(51) Int. Cl. C07C 2/76

(52) U.S. Cl. CPC C07C 2/76; C07C 2529/06

(57) ABSTRACT

Implementations of the disclosed subject matter provide a process for the aromatization of a methane-containing gas stream including contacting the methane-containing gas stream in a reaction zone comprising an aromatization catalyst particulate and a hydrogen acceptor particulate under methane-containing gas aromatization reaction conditions to produce reaction products comprising aromatics and gaseous hydrogen. At least a portion of the gaseous hydrogen produced is bound by the hydrogen acceptor particulate in the reaction zone and removed from the reaction products in the reaction zone. Further, the hydrogen acceptor particulate may be separated from the aromatization catalyst particulate in a separation zone under separation conditions.

### CROSS-REFERENCE TO RELATED APPLICATIONS

This application claims priority to U.S. Provisional Application Ser. No. 62/210,648 filed Aug. 27, 2015, the entire disclosure of which is hereby incorporated by reference. This application also claims priority to U.S. Provisional Application Ser. No. 62/257,424 filed Nov. 19, 2015, the entire disclosure of which is hereby incorporated by reference. This application also claims priority to U.S. Provisional Application Ser. No. 62/257,460 filed Nov. 19, 2015, the entire disclosure of which is hereby incorporated by reference.

### TECHNICAL FIELD OF THE INVENTION

This disclosed subject matter relates to a process for the aromatization of a methane-containing gas stream to form aromatics and hydrogen in a reactor containing both catalyst and hydrogen acceptor particulates in a reactor wherein removal of hydrogen from the reaction zone is accomplished in situ by the hydrogen acceptor, and wherein the catalyst and hydrogen acceptor particulates are subsequently separated in order for each particulate to be regenerated.

### BACKGROUND

The aromatic hydrocarbons (specifically benzene, toluene and xylenes) are the main high-octane bearing components of the gasoline pool and important petrochemical building blocks used to produce high value chemicals and a variety of consumer products, for example, styrene, phenol, polymers, plastics, medicines, and others. Since the late 1930's, aromatics are primarily produced by upgrading of oil-derived feedstocks via catalytic reforming or cracking of heavy naphthas. However, occasional severe oil shortages and oil price spikes result in severe aromatics shortages and aromatics price spikes. Therefore, there is a need to develop new, independent from oil, commercial routes to produce high value aromatics from highly abundant and inexpensive hydrocarbon feedstocks such as methane or stranded natural gas (which typically contains about 80-90% vol. methane).

There are enormous proven reserves of stranded natural gas around the world. According to some estimates, the world reserves of natural gas are at least equal to those of oil. However, unlike the oil reserves that are primarily concentrated in a few oil-rich countries and are extensively utilized, upgraded and monetized, the natural gas reserves are much more broadly distributed around the world and significantly underutilized. Many developing countries that have significant natural gas reserves lack the proper infrastructure to exploit them and convert or upgrade them to higher value products. Quite often, in such situations, natural gas is flared to the atmosphere and wasted. Because of the above reasons, there is enormous economic incentive to develop new technologies that can efficiently convert methane or natural gas to higher value chemical products, specifically aromatics.

In 1993, Wang et al., (Catal. Lett. 1993, 21, 35-41), discovered a direct, non-oxidative route to partially convert methane to benzene by contacting methane with a catalyst containing 2.0% wt. Molybdenum on an H-ZSM-5 zeolite support at atmospheric pressure and a temperature of 700° C. Since Wang's discovery, numerous academic and industrial research groups have become active in this area and have contributed to further developing various aspects of the direct, non-oxidative methane to benzene catalyst and process technology. Many catalyst formulations have been prepared and tested and various reactor and process conditions and schemes have been explored.

Despite these efforts, a direct, non-oxidative methane aromatization catalyst and process cannot yet be commercialized. Some important challenges that need to be overcome to commercialize this process include: (i) the very low, as dictated by thermodynamic equilibrium, per pass conversion and benzene yield (for example, 10% wt. and 6% wt., respectively at 700° C.); (ii) the fact that the reaction is favored by high temperature and low pressure; (iii) the need to separate the produced aromatics and hydrogen from unreacted (mainly methane) hydrocarbon off gas and (iv) the rapid coke formation and deposition on the catalyst surface and corresponding relatively fast catalyst deactivation. Among these challenges, overcoming the thermodynamic equilibrium limitations and significantly improving (e.g., by greater than 3 times) the conversion and benzene yield per pass has the potential to enable the commercialization of an efficient, direct, non-oxidative methane-containing gas aromatization process.

The methane aromatization reaction can be described as follows:

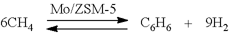

According to the reaction, 6 molecules of methane are required to generate a molecule of benzene. It is also apparent that, the production of a molecule of benzene is accompanied by the production of 9 molecules of hydrogen. Simple thermodynamic calculations revealed and experimental data have confirmed that, the methane aromatization at atmospheric pressure is equilibrium limited to about 10 or 20% wt. at reaction temperatures of 700° C. or 800° C., respectively. In addition, experimental data showed that the above conversion levels correspond to about 6 and 11.5% wt. benzene yield at 700° C. and 800° C., respectively. The aforementioned low methane conversions and benzene yields per pass are not attractive and do not provide an economic justification for scale-up and commercialization of a methane containing gas aromatization process.

Therefore, there is a need to develop an improved direct, non-oxidative methane aromatization process that provides for significantly higher (than those allowed by the thermodynamic equilibrium) methane conversion and benzene yields per pass by implementing an in situ hydrogen removal from the reaction products and the reaction zone.

### BRIEF SUMMARY

According to an embodiment of the disclosed subject matter, a process may include contacting the methane-containing gas stream in a reaction zone comprising an aromatization catalyst particulate and a hydrogen acceptor particulate under methane-containing gas aromatization conditions to produce reaction products comprising aromatics and gaseous hydrogen. At least a portion of the gaseous hydrogen produced is bound by the hydrogen acceptor in the reaction zone and removed from the reaction products in the reaction zone. Next, the hydrogen acceptor particulate may be separated from the aromatization catalyst particulate in a separation zone under separation conditions.

The disclosed subject matter also provides catalyst and/or hydrogen acceptor recycle and regeneration process schemes. According to these schemes, the catalyst and hydrogen acceptor are separated and regenerated separately in separate vessels and then returned to the reactor for continuous (uninterrupted) production of aromatics and hydrogen. The aforementioned in situ hydrogen removal in the reaction zone allows for overcoming the thermodynamic equilibrium limitations by introducing another chemical reaction, between gaseous hydrogen and the hydrogen acceptor particulate. This results in significantly higher and economically more attractive methane-containing gas stream conversion and aromatics yields per pass compared to the process without hydrogen removal, i.e. without hydrogen acceptor particulate in the reaction zone. Further, the disclosed subject matter provides techniques for selecting the catalyst particulate and the hydrogen acceptor particulate for proper mixing and subsequent separation of the two particulates.

Additional features, advantages, and embodiments of the disclosed subject matter may be set forth or apparent from consideration of the following detailed description, drawings, and claims. Moreover, it is to be understood that both the foregoing summary and the following detailed description are examples and are intended to provide further explanation without limiting the scope of the claims.

### BRIEF DESCRIPTION OF THE DRAWINGS

The accompanying drawings, which are included to provide a further understanding of the disclosed subject matter, are incorporated in and constitute a part of this specification. The drawings also illustrate embodiments of the disclosed subject matter and together with the detailed description serve to explain the principles of embodiments of the disclosed subject matter. No attempt is made to show structural details in more detail than may be necessary for a fundamental understanding of the disclosed subject matter and various ways in which it may be practiced.

FIG. 1 shows an example aromatization reactor with catalyst and hydrogen acceptor particulates intermixed in a fluidized bed according to an embodiment of the disclosed subject matter.

FIG. 2 shows a schematic diagram of separation and regeneration of catalyst and hydrogen acceptor particles in separate vessels according to an embodiment of the disclosed subject matter.

FIG. 3 shows an example of two particle size distributions of two example surrogate particulates according to an embodiment of the disclosed subject matter.

FIG. 4 shows an example of the test apparatus demonstrating a condition for mixing the two example surrogate particulates according to an embodiment of the disclosed subject matter.

FIG. 5(a) shows an example of two measured differential pressures under aromatization conditions according to an embodiment of the disclosed subject matter

FIG. 5(b) shows an example of two measured particle size distributions under aromatization conditions according to an embodiment of the disclosed subject matter.

FIG. 6 shows an example of the test apparatus demonstrating a condition for separating the two example surrogate particulates according to an embodiment of the disclosed subject matter.

FIG. 7(a) shows an example of two measured differential pressures under separation conditions according to an embodiment of the disclosed subject matter

FIG. 7(b) shows an example of two measured particle size distributions under separation conditions according to an embodiment of the disclosed subject matter.

FIG. 8 shows an example of transient measurements of upper and lower bed differential pressures upon changing the superficial velocity according to an embodiment of the disclosed subject matter.

FIG. 9 shows example pressure differential measurements at various superficial velocities according to an embodiment of the disclosed subject matter.

FIG. 10(a) shows an example measured particle size distribution at various superficial velocities according to an embodiment of the disclosed subject matter.

FIG. 10(b) shows example measured particle size distribution at various superficial velocities according to an embodiment of the disclosed subject matter.

FIG. 11 shows example pressure differential measurements at various superficial velocities according to an embodiment of the disclosed subject matter.

FIG. 12 shows example measured particle size distribution at a superficial velocity according to an embodiment of the disclosed subject matter.

### DETAILED DESCRIPTION

Methane conversion in a methane aromatization reaction, for example a methane-to-benzene (M2B) reaction is limited by thermodynamic equilibrium. The advantage of using a hydrogen acceptor in the M2B reaction zone is to increase methane conversion by removing hydrogen in situ from the reaction products and hence shift the equilibrium toward higher conversion. In order to achieve such process objectives, the two particulates of hydrogen acceptor and M2B catalyst must be able to mix well together in order to capture and remove hydrogen in situ within the reaction zone.

In general, after the M2B catalyst and hydrogen acceptors are spent, each particulate may need to be regenerated before sending each back to the reaction zone for further reaction processing. Since regeneration conditions for each of the M2B aromatization catalyst and hydrogen acceptor may be different, it is important that the two particulates of hydrogen acceptor and M2B catalyst can be separated from one another in the separation zone in order to enable the two particulates to be regenerated under regeneration conditions, which may be unique to each particulate.

According to the disclosed subject matter, the catalyst particulate and the hydrogen acceptor particulate may be well mixed under reaction conditions in the reaction zone and, subsequently, the particulates may be separated under separation conditions in the separation zone. The disclosed subject matter provides techniques for achieving both well-mixing and separation of the particulates by the novel selection and design of the two particulates in combination with the novel design of the operating conditions in the reaction and separation zones, as disclosed herein. According to the disclosed subject matter, a process for the aromatization of a methane-containing gas stream may include contacting the methane-containing gas stream in a reaction zone comprising an aromatization catalyst particulate and a hydrogen acceptor particulate under methane-containing gas aromatization reaction conditions to produce reaction products comprising aromatics and gaseous hydrogen. At least a portion of the gaseous hydrogen produced is bound by the hydrogen acceptor particulate in the reaction zone and removed from the reaction products in the reaction zone. Further, the process may include separating the hydrogen acceptor particulate from the aromatization catalyst particulate in a separation zone under separation conditions.

The conversion of a methane-containing gas stream to aromatics is typically carried out in an aromatization reactor comprising a catalyst, which is active in the conversion of the methane-containing gas stream to aromatics. The methane-containing gas stream that is fed to the reactor comprises more than 50% vol. methane, more than 60% vol. methane, more than 70% vol. methane and from 75% vol. to 100% vol. methane. The balance of the methane-containing gas may be other alkanes, for example, ethane, propane and butane and other impurity gases. The methane-containing gas stream may be natural gas which is a naturally occurring hydrocarbon gas mixture consisting primarily of methane, with up to about 30% vol. concentration of other hydrocarbons (usually mainly ethane and propane) as well as small amounts of other impurities such as carbon dioxide, nitrogen and others. The methane-containing gas stream may also include recycled unconverted methane which may include products from the aromatization reactions like hydrogen, benzene and naphthalene due to incomplete separation.

Various methane aromatization conditions may be set for carrying out the conversion of the methane-containing gas stream. In general, the conversion of a methane-containing gas stream is carried out at a gas hourly space velocity of from 100 to 60000 h⁻¹, a pressure of from 0.1 to 10 bar(a) and a temperature of from 500 to 900° C. In an embodiment, the conversion is carried out at gas hourly space velocity of from 300 to 30000 h⁻¹, a pressure of from 0.3 to 50 bar(a) and a temperature of from 600 to 875° C. In another embodiment, the conversion is carried out at gas hourly space velocity of from 500 to 10000 h⁻¹, a pressure of from 5 to 25 bar(a) and a temperature of from 650 to 850° C.

Various co-feeds such as CO, CO2 or hydrogen or mixtures thereof that react with coke precursors or prevent their formation during methane aromatization could be added at levels of <10% vol. to the methane-containing feed to improve the stability, performance or regenerability of the catalyst. The methane-containing gas aromatization is then carried out until conversion falls to values that are lower than those that are economically acceptable. At this point, the aromatization catalyst has to be regenerated to restore its aromatization activity to a level similar to its original activity. Following the regeneration, the catalyst is again contacted with a methane-containing gas stream in the reaction zone of the aromatization reactor under aromatization conditions for continuous production of aromatics.

Any catalyst suitable for methane-containing gas stream aromatization may be used in the process of the disclosed subject matter. The catalyst typically comprises one or more active metals deposited on an inorganic oxide support and may optionally comprise promoters or other beneficial compounds. The active metal or metals, promoters, compounds as well as the inorganic support all contribute to the overall aromatization activity, mechanical strength and performance of the aromatization catalyst.

The active metal(s) component of the catalyst may be any metal that exhibits catalytic activity when contacted with a gas stream comprising methane under methane-containing gas aromatization conditions. The active metal may be selected from the group consisting of: vanadium, chromium, manganese, zinc, iron, cobalt, nickel, copper, gallium, germanium, niobium, molybdenum, ruthenium, rhodium, silver, tantalum, tungsten, rhenium, platinum and lead and mixtures thereof. The active metal is preferably molybdenum.

The promoter or promoters may be any element or elements that, when added in a certain preferred amount and by a certain preferred method of addition during catalyst synthesis, improve the performance of the catalyst in the methane-containing gas stream aromatization reaction.

The inorganic oxide support can be any support that, when combined with the active metal or metals and optionally the promoter or promoters contributes to the overall catalyst performance exhibited in the methane aromatization reaction. The support has to be suitable for treating or impregnating with the active metal compound or solution thereof and a promoter compound or solution thereof. The inorganic support preferably has a well-developed porous structure with sufficiently high surface area and pore volume and suitable for aromatization surface acidity. The inorganic oxide support may be one or more of zeolites, non-zeolitic molecular sieves, silica, alumina, zirconia, titania, yttria, ceria, rare earth metal oxides and mixtures thereof. The inorganic oxide support of the disclosed subject matter contains zeolite as the primary component. The zeolite may be a ZSM-5, ZSM-22, ZSM-8, ZSM-11, ZSM-12 or ZSM-35 zeolite structure types. The zeolite is preferably a ZSM-5 zeolite. The ZSM-5 zeolite further may have a SiO₂/Al₂O₃ ratio of 10 to 100 mass/mass. Preferably, the SiO₂/Al₂O₃ ratio of the zeolite is in the range of 20-50. Even more preferably the SiO₂/Al₂O₃ ratio is from 20 to 40 and most preferably about 30. The support may optionally contain about 15-70% wt. of a binder that binds the zeolite powder particles together and allows for shaping of the catalyst in the desired form and for achieving the desired high catalyst mechanical strength necessary for operation in a commercial aromatization reactor. More preferably the support contains from 15-30% wt. binder. The binder is selected from the group consisting of silica, alumina, zirconia, titania, yttria, ceria, rare earth oxides or mixtures thereof.

The aromatization catalyst particulate may be in the form of cylindrical pellets, rings, spheres, and the like. As an example, in a fluidized bed reactor operation, the catalyst may be a particulate material comprising particles, and each particle shape may be spherical. The spherical catalyst particulate could be prepared by any method known to those skilled in the art. Preferably, the spherical catalyst may be prepared via spray drying of zeolite containing sols of appropriate concentration and composition. The zeolite containing sol may optionally contain binder. The spherical catalyst particle may have a predominant particle size or diameter that makes it suitable for a particular reactor type, such as a fluidized bed reactor. The spherical particle diameter of the catalyst is preferably selected to be in the range of 1-200 microns. More preferably, the spherical catalyst may have a particle diameter in the range of 20 to 120 microns, and preferably an average particle size of 70 to 80 microns. In general, approximately 95% of the aromatization catalyst particles may fall within the size ranges provided herein.

According to an implementation of the disclosed subject matter, the methane-containing gas stream conversion and corresponding benzene yield per pass are higher than the conversion and yield obtained with the same aromatization catalyst and under the same methane-containing gas aromatization conditions, but in the absence of a hydrogen acceptor in the reaction zone of the aromatization reactor. The hydrogen acceptor used in this reaction can be any metal-containing alloy or a compound that has the ability, when subjected to aromatization operating conditions, to selectively accept or react with hydrogen to form a sufficiently strong hydrogen-acceptor bond. The hydrogen acceptor preferably reversibly binds the hydrogen in such a way that during operation in the aromatization reactor the hydrogen is strongly bound to the acceptor under the methane-containing gas stream aromatization conditions. In addition, the hydrogen acceptor is preferably able to release the hydrogen when transported to the regeneration section where it is subjected to a different set of (regeneration) conditions that favor release of the previously bound hydrogen and regeneration of the hydrogen acceptor. The hydrogen acceptor could be a particle(s) in the form of cylindrical pellets, rings, spheres, a monolithic structure, a porous net-shaped structure, and the like. According to an implementation of the disclosed subject matter, the hydrogen acceptor particulate may include a plurality of particles, each particle having a particle size in the range of 100-2000 microns. More preferably, the hydrogen acceptor particulate may have a particle diameter in the range of 200 to 1500 microns, and preferably with an average particle size of 500 to 1000 microns. In general, approximately 95% of the hydrogen acceptor particles may fall within the size ranges provided herein.

Suitable hydrogen acceptors metals include: Ti, Zr, V, Nb, Hf, Mg, La, Th, Sc as well as other transition metals, elements or compounds or mixtures thereof. The hydrogen acceptor may comprise metals that exhibit magnetic properties, such as for example Fe, Co or Ni or various ferro-, para- or dimagnetic alloys of these metals. One or more hydrogen acceptors that exhibit appropriate particle sizes and mass for fluidized bed aromatization operation may be used in the reaction zone to achieve the desired degree of hydrogen separation and removal.

The mixing of both types of particles, i.e., catalyst particles and hydrogen acceptor particles, provides for the quick removal of the produced hydrogen from the reaction zone and for shifting the aromatization reaction equilibrium toward greater methane-containing gas conversion and benzene yields per pass. This mixing of both types of particles can be achieved in a variety of aromatization reactor configurations. According to an embodiment of the disclosed subject matter, the aromatization reactor may be a fluidized bed reactor. Based on the reactor utilized, the size, shape, and arrangement of the hydrogen acceptor and/or catalyst particulates may be selected to maximize the efficiency of the aromatization reaction and process conditions. Yet another advantage of the presently disclosed subject matter is that the shapes, sizes and mass of both the hydrogen acceptor and the aromatization catalyst may be designed and selected in such a way so that the particulates can be co-fluidized in the aromatization reactor to form a well-mixed fluidized bed. Also, the disclosed subject matter provides for two or more different hydrogen acceptors (e.g., different by chemical formula and/or physical properties) to be simultaneously used with the aromatization catalyst in the aromatization reactor to achieve the desired degree of hydrogen separation from the aromatization reaction zone.

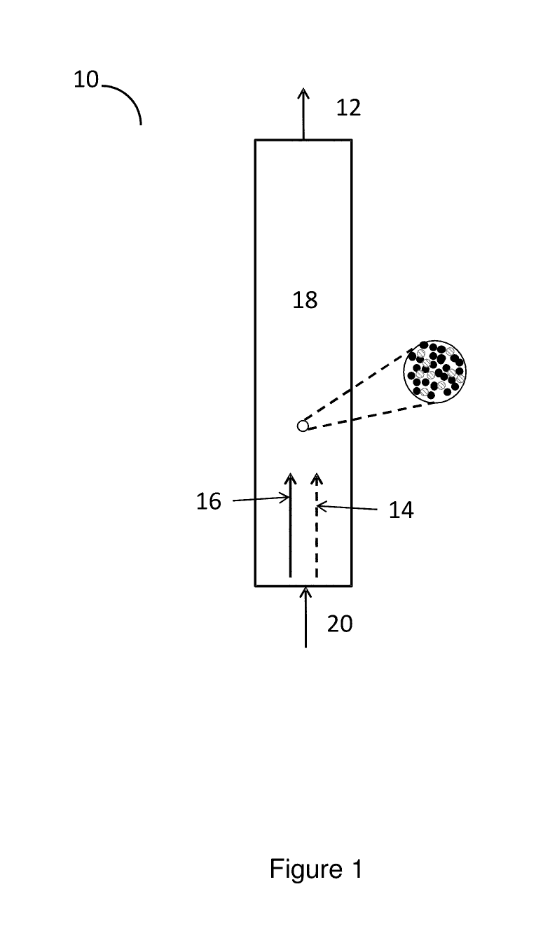

The aromatization reaction of the disclosed subject matter is carried out in an aromatization reactor. To enable this, a suitably shaped and sufficiently robust catalyst and hydrogen acceptor are used for the reaction. A significant advantage of the process of the disclosed subject matter is that it provides for in situ removal of produced hydrogen from the reaction products and reaction zone. As a result, the disclosed subject matter results in a significant increase of both methane-containing gas stream conversion and benzene yield per pass to values that are significantly higher relative to these dictated by the methane aromatization reaction equilibrium. This is enabled by mixing and/or placing the catalyst and hydrogen acceptor particulates in a fluidized-bed state in the reaction zone of the aromatization reactor (e.g., see FIG. 1). For example, as shown in FIG. 1, a fluidized bed reactor 10 comprises a mixture of catalyst and hydrogen acceptor particulates in the fluidized bed 18. The methane-containing gas stream, the catalyst and hydrogen acceptors are introduced via one or more inlets 20 and the products, unreacted gases, catalyst and hydrogen acceptor particulates are removed from the bed via one or more outlets 12. The feed and product generally flow in an upward direction, indicated by arrow 16. The catalyst and hydrogen acceptor are well mixed and generally flow in an upward direction, indicated by arrow 14.

An important feature of the presently disclosed subject matter is the selection of an aromatization catalyst particulate and a hydrogen acceptor particulate that allows for mixing of the two particulates in the reaction zone and subsequent separation of the two particulates in the separation zone. The selection and/or design of the aromatization catalyst particulate and the hydrogen acceptor particulate may be based on a physical property such as the minimum fluidization velocity of each particulate. A minimum fluidization velocity is the minimum gas flow rate at which the particulate becomes fluidized, i.e., the minimum gas velocity required to fluidize a packed bed of particles. According to an embodiment, the aromatization catalyst particulate may have a first set of physical properties including a first minimum fluidization velocity. Similarly, the hydrogen acceptor particulate may have a second set of physical properties comprising a second minimum fluidization velocity. In an embodiment, the first minimum fluidization velocity may be different from the second minimum fluidization velocity, i.e., the minimum fluidization velocity of the aromatization catalyst particulate may be different from the minimum fluidization velocity of the hydrogen acceptor particulate.

As mentioned above, an important feature of the presently disclosed subject matter is that the two particulates may be well-mixed in the reaction zone and may be subsequently separated from one another in the separation zone (i.e., no longer well-mixed). In general, well-mixed may indicate that the two particulates are homogeneously distributed within the reaction zone. In general, separation of the two particulates may indicate that the two particulates are separated in two distinctive phases, for example, one phase above the other phase. This significant advantage may be achieved based on the relative difference between the minimum fluidization velocity of the aromatization catalyst particulate as compared to the minimum fluidization velocity of the hydrogen acceptor particulate. In order to achieve well-mixing of the aromatization catalyst particulate and the hydrogen acceptor particulate in the reaction zone, according to an embodiment, the ratio of the second minimum fluidization velocity (e.g., of the hydrogen acceptor particulate) to the first minimum fluidization velocity (e.g., of the aromatization catalyst particulate) may be less than 200. Similarly, according to an embodiment, the ratio of the first minimum fluidization velocity (e.g., of the aromatization catalyst particulate) to the second minimum fluidization velocity (e.g., of the hydrogen acceptor particulate) may be less than 200. For example, the hydrogen acceptor particulate may have a minimum fluidization velocity 0.46 ft/sec and the aromatization catalyst particulate may have a minimum fluidization velocity of 0.008 ft/sec. In this case, the ratio of the minimum fluidization velocity of the hydrogen acceptor to the minimum fluidization velocity of the aromatization catalyst is 57.5 (i.e., 0.46 ft/sec:0.008 ft/sec=57.5). Accordingly, because this ratio of 57.5 is less than 200, the two particulates may be well-mixed.

In order to achieve separation of the aromatization catalyst particulate and the hydrogen acceptor particulate in the separation zone, according to an embodiment, the ratio of the second minimum fluidization velocity (e.g., of the hydrogen acceptor particulate) to the first minimum fluidization velocity (e.g., of the aromatization catalyst particulate) may be more than 15. Similarly, according to an embodiment, the ratio of the first minimum fluidization velocity (e.g., of the aromatization catalyst particulate) to the second minimum fluidization velocity (e.g., of the hydrogen acceptor particulate) may be more than 15. For example, the hydrogen acceptor particulate may have a minimum fluidization velocity of 0.46 ft/sec and the aromatization catalyst particulate may have a minimum fluidization velocity of 0.008 ft/sec. In this case, the ratio of the minimum fluidization velocity of the hydrogen acceptor to the minimum fluidization velocity of the aromatization catalyst is 57.5 (i.e., 0.46 ft/sec:0.008 ft/sec=57.5). Accordingly, because this ratio of 57.5 is more than 15, the two particulates may be separated.

The aromatization reaction conditions and the separation conditions may include a superficial velocity, among other parameters as described herein (e.g., temperature, pressure, feed rate, and the like). Superficial velocity is a flow velocity calculated as if the given fluid were the only one flowing in a given cross sectional area of the vessel, and may be expressed in any suitable format such as m/s, ft/s, and the like. In an embodiment, the superficial velocity under aromatization reaction conditions and under separation conditions may be selected based on the greater minimum fluidization velocity between the minimum fluidization velocity of each of the hydrogen acceptor particulate and aromatization catalyst particulate. According to an embodiment, the second minimum fluidization velocity may be greater than the first minimum fluidization velocity. In this case, the aromatization reaction conditions may include a superficial velocity that is greater than 1.5 times the second minimum fluidization velocity. Similarly, according to an implementation, the second minimum fluidization velocity may be greater than the first minimum fluidization velocity, and in this case, the separation conditions may include a superficial velocity that is less than 1.5 times the second minimum fluidization velocity. For example, the hydrogen acceptor particulate may have a minimum fluidization velocity 0.46 ft/sec and the aromatization catalyst particulate may have a minimum fluidization velocity of 0.008 ft/sec. In this case, the minimum fluidization velocity of the hydrogen acceptor particulate is greater than the minimum fluidization velocity of the aromatization catalyst (i.e., 0.46 ft/sec>0.008 ft/sec). Accordingly, the aromatization conditions may include a superficial velocity that is greater than 1.5 times the minimum fluidization velocity of the hydrogen acceptor. In particular, the aromatization conditions may include a superficial velocity of 1.2 ft/sec which is greater than 0.69 ft/sec (i.e., 1.5 times 0.46 ft/sec=0.69 ft/sec). In this case, with a superficial velocity of 1.2 ft/sec, the two particulates are well-mixed in the reaction zone. Furthermore, the separation conditions may include a superficial velocity of 0.49 ft/sec which is less than 1.5 times the minimum fluidization velocity of the hydrogen acceptor. In particular, the separation conditions may include a superficial velocity of 0.49 ft/sec which is less than 0.69 ft/sec (i.e., 1.5 times 0.46 ft/sec=0.69 ft/sec). In this case, with a superficial velocity of 0.49 ft/sec, the two particulates are separated in the separation zone.

In an alternative embodiment, the first minimum fluidization velocity may be greater than the second minimum fluidization velocity. In this case, the aromatization conditions may include a superficial velocity that is greater than 1.5 times the first minimum fluidization velocity. Similarly the first minimum fluidization velocity may be greater than the second minimum fluidization velocity. Accordingly, the separation conditions may include a superficial velocity that is less than 1.5 times the first minimum fluidization velocity.

The separation conditions may further include a particulate residence time, which may be different from the gas residence time. The particulate residence time may be the average amount of time that both particulates spend in the separation zone. In an embodiment, the particulate residence time may be more than 10 seconds. In contrast, the gas residence time may be the average time the reacting gasses remain in the reaction zone. For example, this may be based on the volume of the incoming feed gas, the volume of the product gasses, and/or an average thereof. The gas residence may or may not also account for the volume of the catalyst and/or hydrogen acceptor particulates. In an implementation, the separation zone may be located in a separation vessel or in a separation zone of a reactor vessel, and in some cases, the reactor vessel may also be the separation vessel.

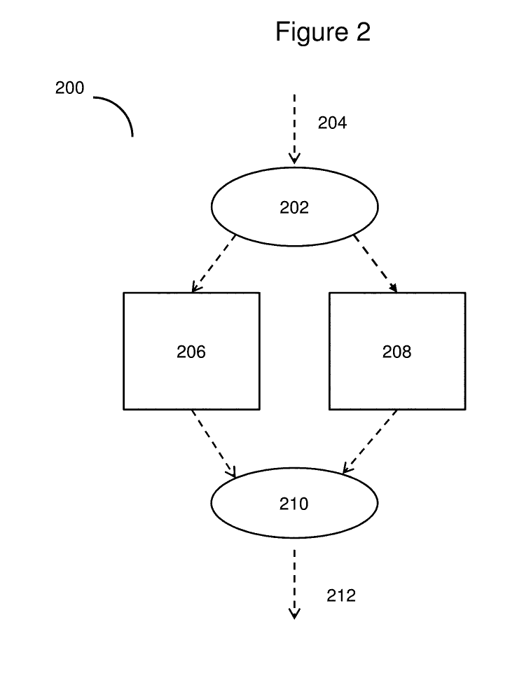

An important advantage of the process of this invention is that it provides for the aromatization catalyst and the hydrogen acceptor to be separated and withdrawn from the reaction zone of the aromatization reactor and regenerated. According to an implementation, the process may further provide for continuously regenerating the catalyst to remove coke formed during the reaction and continuously regenerating the hydrogen acceptor by releasing the hydrogen under regeneration conditions. In an implementation, the catalyst and hydrogen acceptor may be regenerated in separate vessels. As an example, the aromatization catalyst and hydrogen acceptor may be regenerated in separate vessels according to the example scheme illustrated in FIG. 2 and then continuously returned back to the aromatization reactor for continuous production of aromatics and hydrogen. The hydrogen acceptor and catalyst regeneration could be accomplished simultaneously, stepwise, or separately in separate vessels as illustrated in FIG. 2. This operation scheme provides for maximum flexibility to accomplish the hydrogen release or regeneration of the acceptor and catalyst under different and suitable set of regeneration conditions, which may be unique to each particulate. The regeneration of catalyst and hydrogen acceptor could be accomplished in fixed, moving or fluidized bed reactor vessels schematically shown in FIG. 2.

As mentioned above, FIG. 2 shows a schematic diagram of separation and regeneration of catalyst and hydrogen acceptor particles in separate vessels according to an embodiment of the disclosed subject matter. According to an implementation, the process disclosed herein may also include continuously regenerating the catalyst to remove coke formed during the reaction under first regeneration conditions in a first regeneration vessel. Similarly, in an embodiment, the disclosed process may also include continuously regenerating the hydrogen acceptor by releasing the hydrogen under second regeneration conditions in a second regeneration vessel. As shown for example in FIG. 2, the aromatization catalyst particulate and hydrogen acceptor particulate may each be regenerated under different regeneration conditions. In FIG. 2, regenerator system 200 may comprise a separation zone 202 under separation condition to separate the aromatization catalyst particulate from the hydrogen acceptor particulate that is fed from the reactor via line 204. This separation zone 202 may be the process according to the disclosed subject matter. The aromatization catalyst particulate may be fed to catalyst regeneration vessel 206, and the hydrogen acceptor particulate may be fed to hydrogen acceptor regeneration vessel 208. The regenerated aromatization catalyst particulate and hydrogen acceptor particulate may then be mixed back together in mixing step 210 and then fed back to the reactor via line 212. In an embodiment, the regenerated aromatization catalyst particulate and hydrogen acceptor particulate may be fed back to the reactor via line 212 without the mixing step 210.

It is well known that the methane-containing gas aromatization catalysts form coke during the reaction. Accumulation of coke on the surface of the catalyst gradually covers the active aromatization sites of the catalyst resulting in gradual reduction of its activity. Therefore, the coked catalyst has to be removed at certain carefully chosen frequencies from the reaction zone of the aromatization reactor and regenerated in a regeneration vessel as depicted in FIG. 2. The regeneration of the catalyst can be carried out by any method known to those skilled in the art. For example, two possible regeneration methods are hot hydrogen stripping and oxidative burning at temperatures sufficient to remove the coke from the surface of the catalyst. If hot hydrogen stripping is used to regenerate the catalyst, then at least a portion of the hydrogen used for the catalyst regeneration may come from the hydrogen released from the hydrogen acceptor. Additionally, fresh hydrogen may be fed to the catalyst regeneration vessel as needed to properly supplement the hydrogen released from the hydrogen acceptor and to complete the catalyst regeneration. If the regeneration of the two particulates is carried out in different vessels (e.g., see FIG. 2) the operating conditions of each vessel could be selected and maintained to favor the regeneration of the catalyst or the hydrogen acceptor. Hydrogen removed from the hydrogen acceptor could then again be used to at least partially hydrogen strip and regenerate the catalyst.

Yet another important advantage of the process of the disclosed subject matter over the prior art is that it provides for the release of the hydrogen that is bound to the hydrogen acceptor when the saturated, or partially saturated, acceptor is subjected to a specific set of conditions in the regeneration vessel(s). Furthermore, the released hydrogen could be utilized to regenerate the catalyst or subjected to any other suitable chemical use or monetized to improve the overall aromatization process economics.

Another important advantage of the disclosed subject matter is that it allows for different regeneration conditions to be used in the different regeneration vessel or vessels to optimize and minimize the regeneration time required for the catalyst and hydrogen acceptor and to improve performance in the aromatization reaction.

### Examples

### Design of the Two Particulates

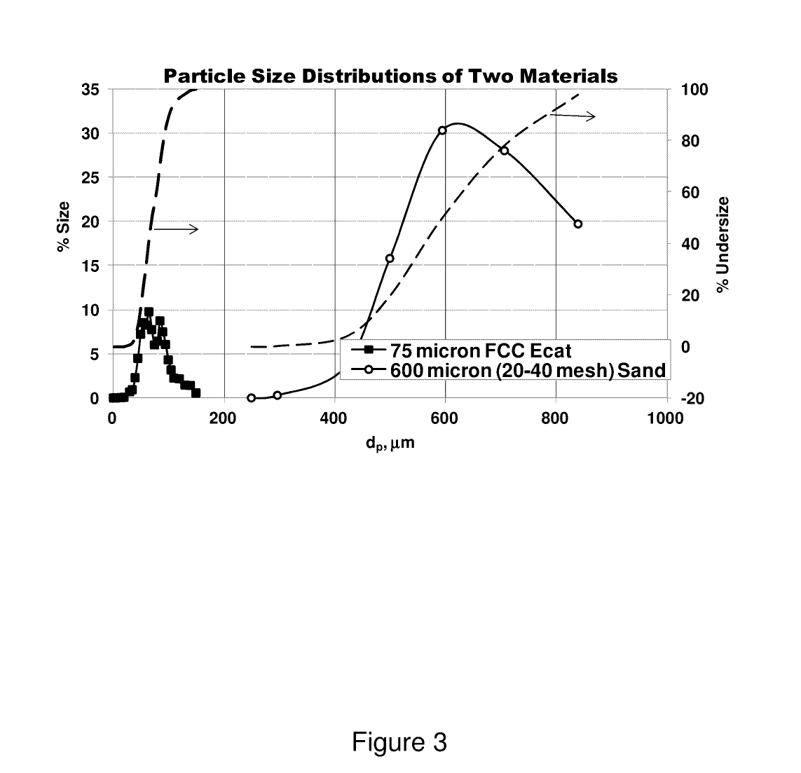

The following example demonstrates the design of the two particulates according to the disclosed subject matter. Since the mixing and separation of the two particulates are pure physical processes, the following example utilized readily available surrogate particulate materials to simulate a M2B aromatization catalyst particulate and a hydrogen acceptor particulate. The particle size distributions (PSDs) of the two surrogate materials are shown in FIG. 3. In the example, the smaller, less dense (e.g., lighter) particles were equilibrium catalyst (E-cat) from a refinery Fluid Catalytic Cracking (FCC) unit. These particles had an average diameter of about 75 microns with a particle size distribution ranging from about 0.5 microns to about 160 microns. The minimum fluidization velocity of this FCC E-cat particulate with ambient condition air is about 0.008 ft/sec. In the example, the larger and denser particles were common sand. The average size of these particles is about 500 microns, with a particle size distribution ranging from 200 microns to 1,000 microns. The minimum fluidization velocity of this sand particulate with ambient condition air is about 0.46 ft/sec. According to an aspect of the disclosed subject matter, the ratio of the minimum fluidization velocity of the sand particulate to the minimum fluidization velocity of the FCC E-cat particulate is 57.5 (i.e., 0.46 ft/sec:0.008 ft/sec). As such, this ratio of 57.5 is less than 200 and this ratio of 57.5 is greater than 15, according to the disclosed subject matter.

Demonstration of Well-Mixing of the Two Surrogate Particulates Under Reaction Conditions in the Reaction Zone:

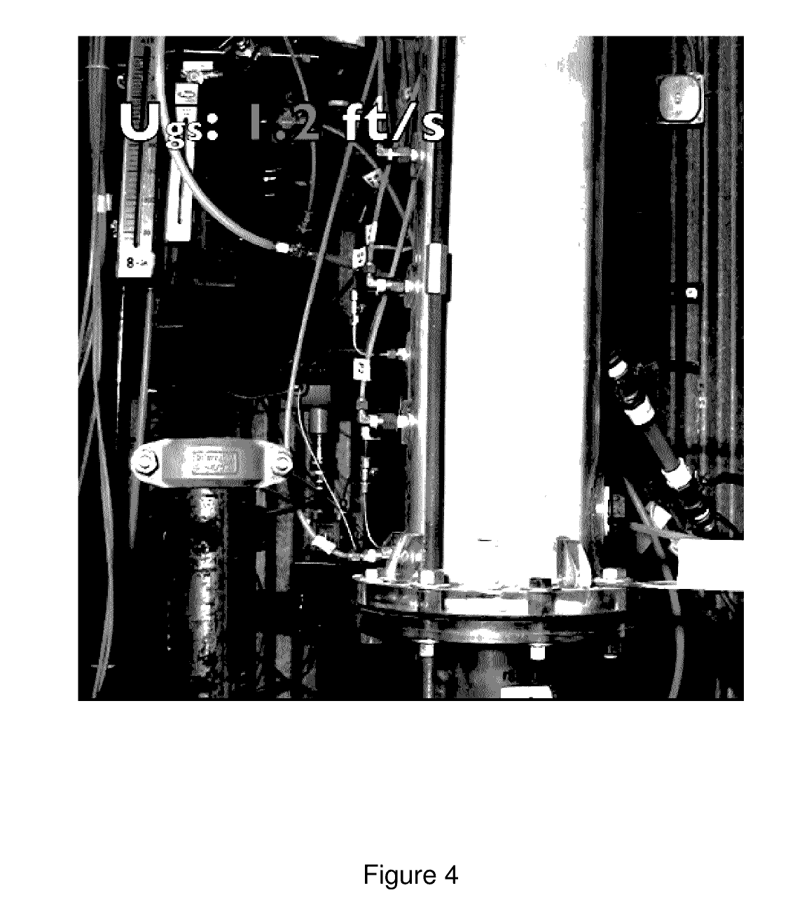

For purposes of the examples provided herein, air was used as a surrogate gas to simulate the methane-containing feed gas in the reaction zone or the feed gas (or inert gas) in the separation zone. With a superficial air velocity of 1.2 ft/sec, which is well above the heavier particle minimum fluidization velocity of approximately 0.46 ft/sec (i.e., sand particulate), the E-cat and sand particulates are visually well mixed, as shown in FIG. 4. As an example, the minimum fluidization velocity of the sand particulate is 0.46 ft/sec which is greater than the minimum fluidization velocity of the FCC E-cat particulate of 0.008 ft/sec. Accordingly, the aromatization reaction conditions include a superficial velocity of 1.2 ft/sec which is greater than 1.5 times the minimum fluidization velocity of the sand particulate which is 0.46 ft/sec. In particular, 1.5*0.46 ft/sec=0.69 ft/sec, and the superficial velocity under aromatization conditions of 1.2 ft/sec is greater than 0.69 ft/sec).

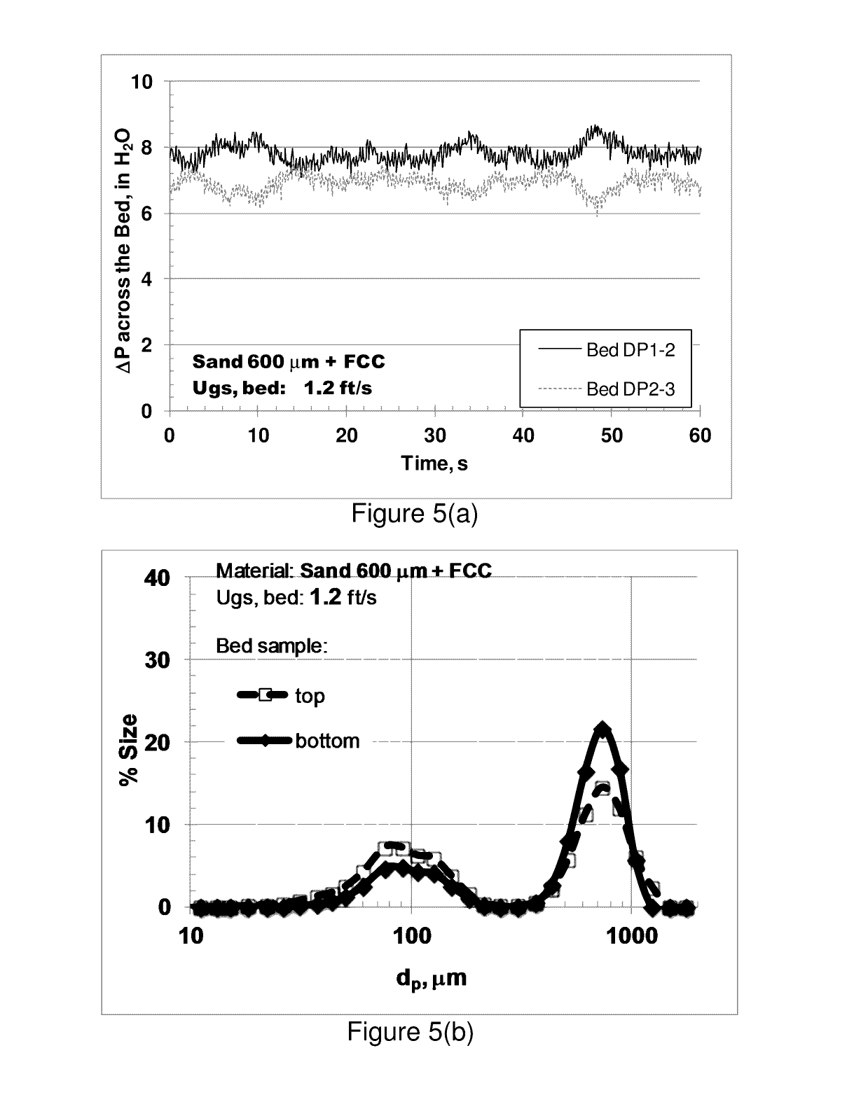

Turning to FIGS. 5(a) and 5(b), the two particulate samples and pressure differential measurements from the upper and lower bed sections also confirms that the two particulates are well-mixed in the reaction zone. FIG. 5(a) shows an example of measured differential pressures under aromatization conditions including a superficial air velocity of 1.2 ft/sec (i.e., fluidization velocity). The measured differential pressure for the upper section of the bed is depicted by the solid line (i.e. Bed DP1-2) and the lower section of the bed as depicted by the dashed line (i.e., Bed DP2-3). As can be seen in FIG. 5(a), the pressure differential measurement taken at the top and bottom of the bed are very similar, indicating that the particulates are well-mixed. FIG. 5(b) shows measured particle size distributions based on bed samples taken at top and bottom locations of the bed under aromatization conditions including a superficial air velocity of 1.2 ft/sec (i.e., fluidization velocity). As shown, the measured particle size distribution depicted by open-square line markers was taken at the location of the top layer and the measured particle size distribution depicted by solid-diamond shaped line markers was taken at the location of the bottom layer of the bed. As can be seen in FIG. 5(b), the measured particle size distributions are very similar at both the top and bottom locations of the bed. This confirms that the two particulate samples are well-mixed under aromatization conditions including a superficial air velocity of 1.2 ft/sec (i.e., fluidization velocity).

Demonstration of Separation of the Two Surrogate Particulates Under Separation Conditions in the Separation Zone:

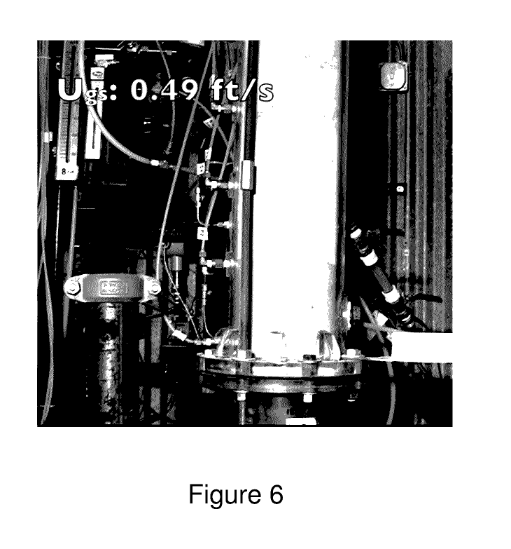

FIG. 6 shows an example test apparatus demonstrating separation according to an embodiment of the disclosed subject matter. At a superficial air velocity of 0.49 ft/sec, which is slightly higher than the minimum fluidization velocity of the heavier particles of 0.46 ft/sec, the two particulates are visually separated, with the larger/heavier sand particles in the lower section and smaller/lighter E-cat in the upper section, as shown in FIG. 6. As an example, the minimum fluidization velocity of the sand particulate is 0.46 ft/sec which is greater than the minimum fluidization velocity of the FCC E-cat particulate of 0.008 ft/sec. Accordingly, the separation conditions include a superficial air velocity of 0.49 ft/sec which is less than 1.5 times the minimum fluidization velocity of the sand particulate which is 0.46 ft/sec. In particular, 1.5*0.46 ft/sec=0.69 ft/sec, and the superficial velocity under separation conditions of 0.49 ft/sec is less than 0.69 ft/sec).

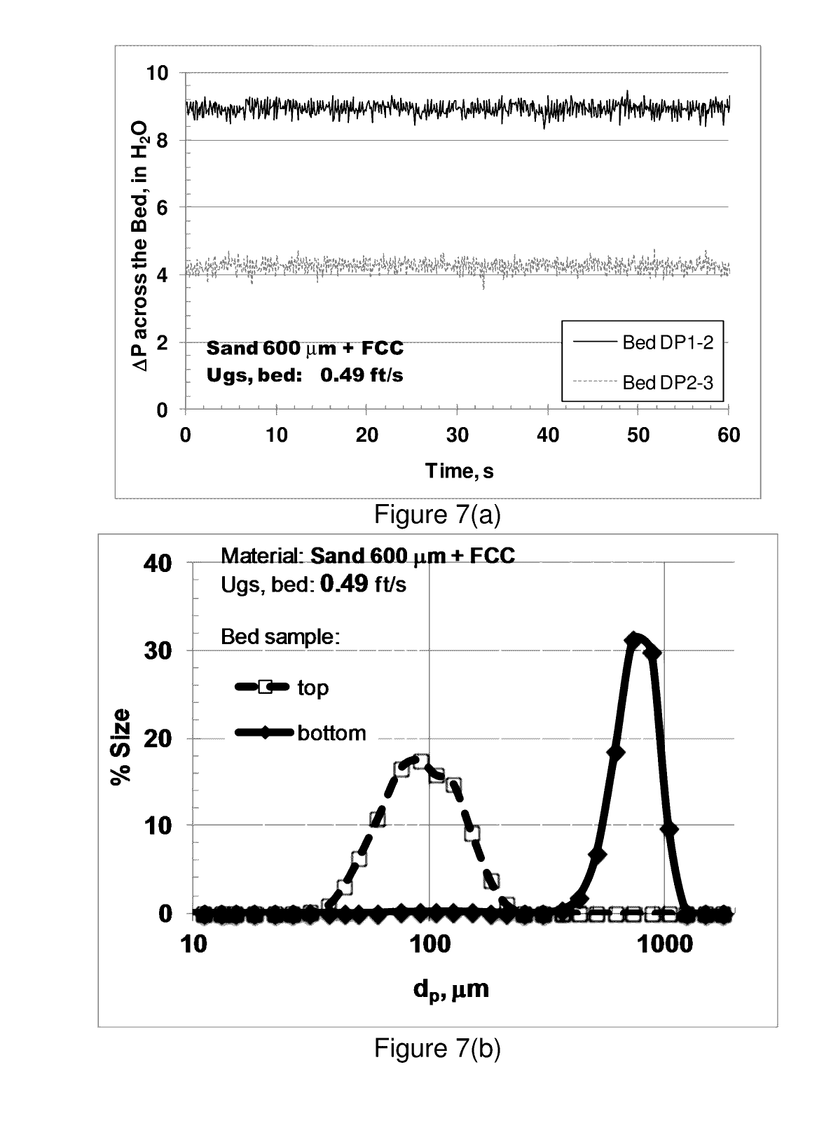

Turning to FIGS. 7(a) and 7(b), the two particulate samples and pressure differential measurements from the upper and lower bed sections also confirm that the two particulates are indeed separated. FIG. 7(a) shows an example of two measured differential pressures under separation conditions including a superficial velocity of 0.49 ft/sec. The measured differential pressure for the upper section of the bed is depicted by the solid line (i.e. Bed DP1-2) and the lower section of the bed as depicted by the dashed line (i.e., Bed DP2-3). As can be seen from FIG. 7(a), the differential pressures measured at the top and bottom of the bed are very different from one another, demonstrating the separation of the two surrogate particulates according to an embodiment of the disclosed subject matter. FIG. 7(b) shows measured particle size distributions based on bed samples taken at top and bottom locations of the bed under separation conditions including a superficial air velocity of 0.49 ft/sec. As shown, the measured particle size distribution depicted by open-square line markers was taken at the location of the top layer and the measured particle size distribution depicted by solid-diamond shaped line markers was taken at the location of the bottom layer of the bed. As can be seen in FIG. 7(b), the measured particle size distributions are very different at the top and bottom locations of the bed. Based on the two very different measured particle size distributions at the top and bottom of the bed under separation conditions, this demonstrates successful separation of the two surrogate particulates according to an embodiment of the disclosed subject matter. This confirms that the two particulates are separated under separation conditions including a superficial air velocity of 0.49 ft/sec.

Demonstration of Time Requirement in the Separation Zone to Achieve Separation:

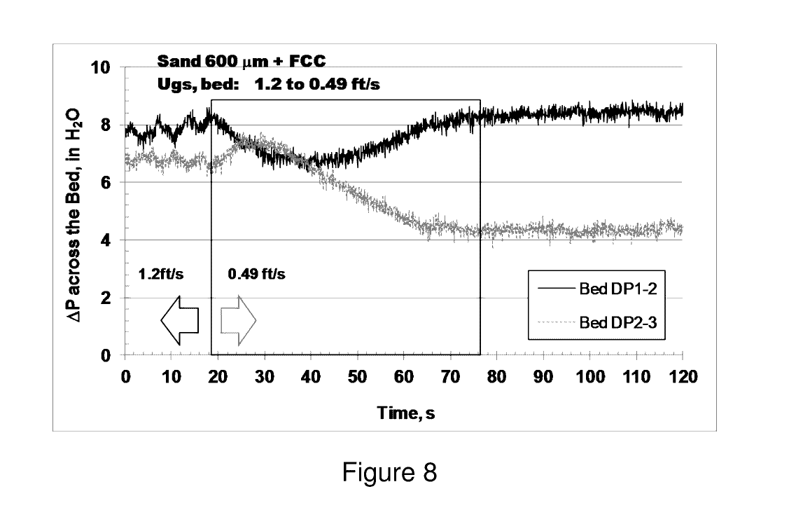

When subjecting the well-mixed two particulates under separation condition in the separation zone, a certain amount of time (e.g., particulate residence time) is required to achieve the desired separation of the two particulates, as shown in FIG. 8. The measured differential pressure for the upper section of the bed is depicted by the solid line (i.e. Bed DP1-2) and the lower section of the bed as depicted by the dashed line (i.e., Bed DP2-3). As shown in FIG. 8, the two pressure differential measurements from upper and lower locations in the bed are very similar at a superficial velocity of 1.2 ft/sec, indicating that the two particulates are well-mixed. The superficial velocity air flow is changed from 1.2 ft/sec to 0.49 ft/sec to initiate separation under the separation conditions in the separation zone around the 19 second point in time. As can be seen, separation of the two particulates does not occur immediately; instead, it takes about 50 seconds in this transition test to achieve the desired separation as shown in FIG. 8. In particular, and in accordance with the disclosed subject matter, when the separation conditions include a particulate residence time that is more than 10 seconds, the two particulates may be separated. As such, in the example, the particulate residence time of 50 seconds in the separation zone is more than 10 seconds and achieves the desired separation of the two particulates.

Additional Examples of the Design of the Two Particulates:

The following two examples demonstrate the significance of the design of the two particulates according to the disclosed subject matter. The first example demonstrates that if the two particulates have minimum fluidization velocities that are too similar, they may not be separated. In particular, if the ratio of one minimum fluidization velocity to the other minimum fluidization velocity is not more than 15, the two particulates may not be separated. The second example demonstrates that if the two particulates have fluidization velocities that are too dissimilar, the two particulates may not be well-mixed. Specifically, if the ratio of one minimum fluidization velocity to the other minimum fluidization velocity is not less than 200, the two particulates may not be well-mixed.

The first example uses the same FCC E-cat surrogate aromatization catalyst particulate and a finer sand particulate representing a surrogate hydrogen acceptor particulate having an average size of 185 microns. The minimum fluidization velocity of this finer sand particulate with ambient condition air is about 0.1 ft/sec as compared to the minimum fluidization velocity of the FCC E-cat particulate of 0.008 ft/sec. In particular, the ratio of the minimum fluidization velocity of the finer sand particulate (i.e., 0.1 ft/sec) to the minimum fluidization velocity of the FCC E-cat particulate (i.e., 0.008 ft/sec) is 12.5 (i.e., 0.1 ft/sec:0.008 ft/sec=12.5). This ratio of 12.5 is less than 200 in accordance with the presently disclosed subject matter. However, contrary to the presently disclosed subject matter, 12.5 is not more than 15. As such, the two particulates may be well-mixed, but may not be successfully separated.

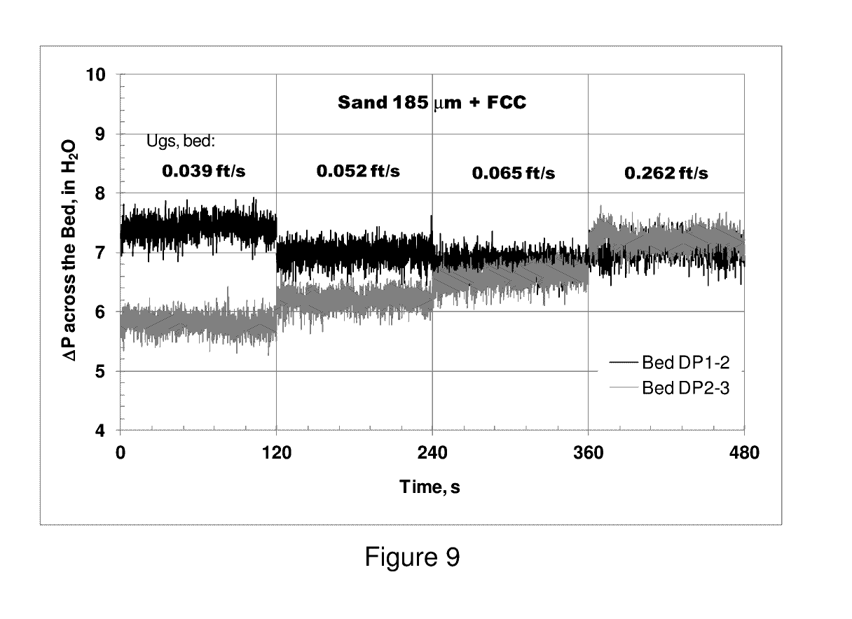

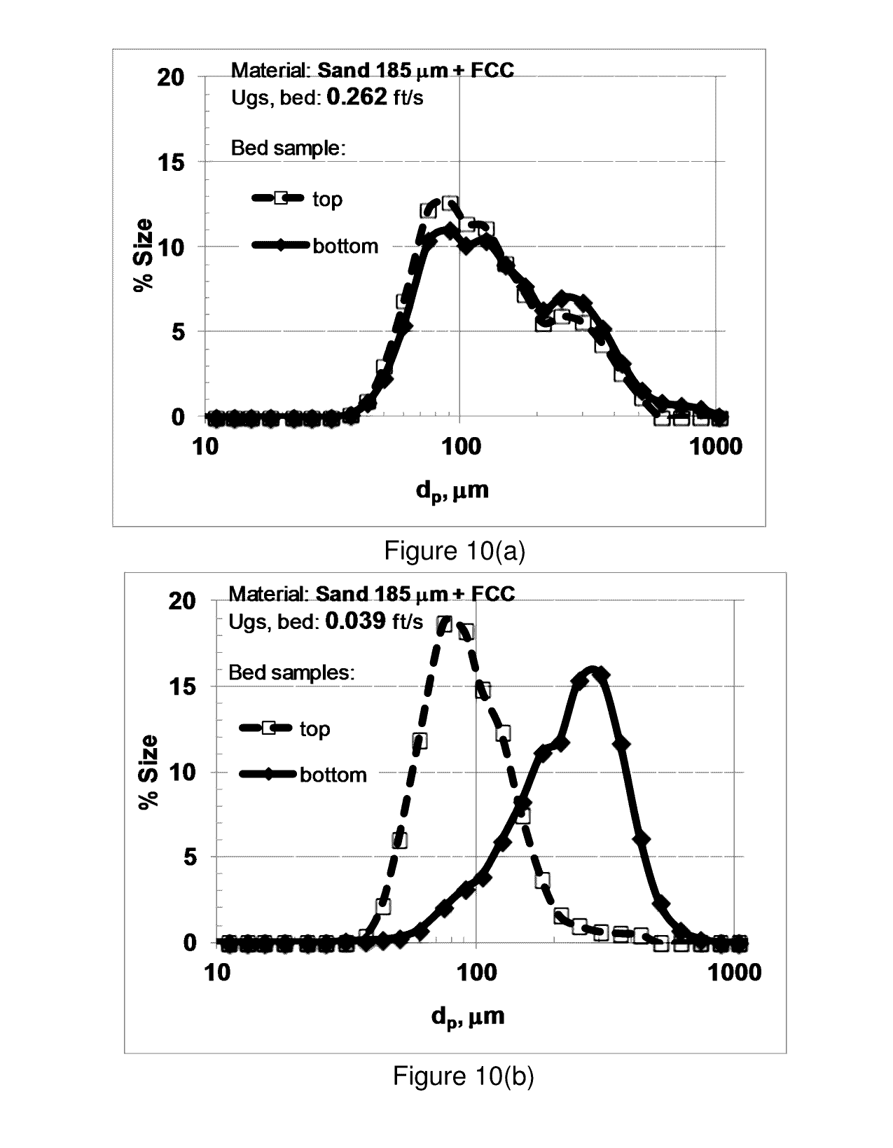

FIG. 9 shows the pressure differential measurement in the upper and lower locations of the test bed at different superficial velocities, indicating that the two particulates appear to be well-mixed at a superficial velocity of 0.262 ft/sec based on the two similar pressure differential measurements. Accordingly, this confirms that because the ratio of 12.5 (i.e., the ratio of the minimum fluidization velocity of the finer sand particulate to the minimum fluidization velocity of the FCC E-cat particulate) is less than 200, the two particulates are well-mixed as shown by the pressure differential measurements provided in FIG. 9. The additional measurements of direct samplings from the upper and lower sections of the bed demonstrate that the two particulates are indeed well-mixed at a superficial velocity of 0.262 ft/sec, shown in FIG. 10(a), with very similar particle size distributions. However, contrary to the present invention, because the ratio of 12.5 (i.e., the ratio of the minimum fluidization velocity of the finer sand particulate to the minimum fluidization velocity of the FCC E-cat particulate) is not more than 15, the two particulates cannot be substantially separated. This is shown in FIG. 10(b). As shown in FIG. 10(b), the measured particle size distribution depicted by open-square line markers was taken at the location of the top layer and the measured particle size distribution depicted by solid-diamond shaped line markers was taken at the location of the bottom layer of the bed. In FIG. 10(b), the two particulates are not substantially separated at a superficial velocity of 0.039 ft/sec as shown by the substantial overlap of the two particle size distributions. This confirms that because the ratio of 12.5 (i.e., the ratio of the minimum fluidization velocity of the finer sand particulate to the minimum fluidization velocity of the FCC E-cat particulate) is not more than 15, the two particulates are not substantially separated. This clearly demonstrates that when the minimum fluidization velocities of the two particulates are too similar (i.e., when the ratio of one minimum fluidization velocity to the other minimum fluidization velocity is not more than 15), the particulates may not be separated.

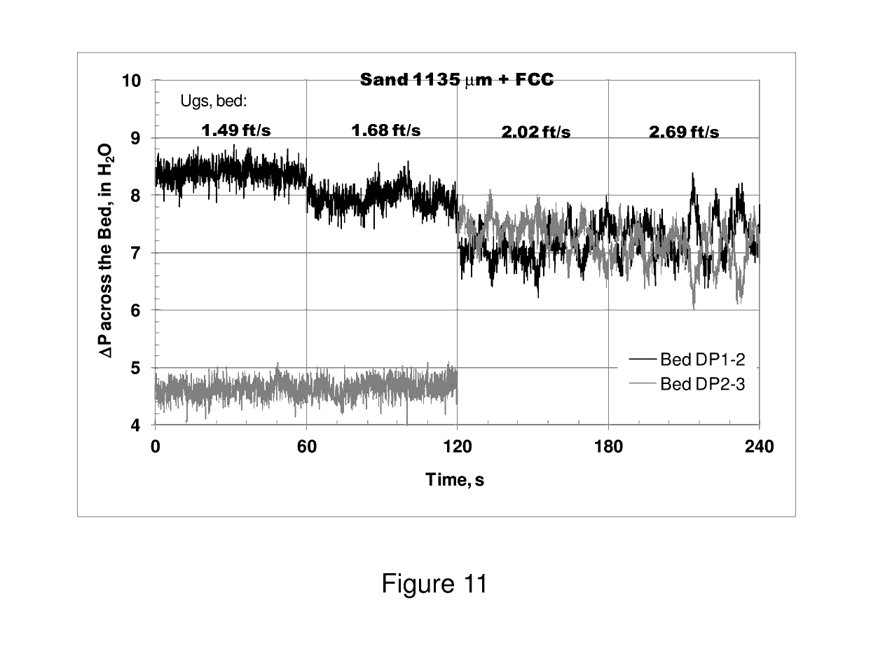

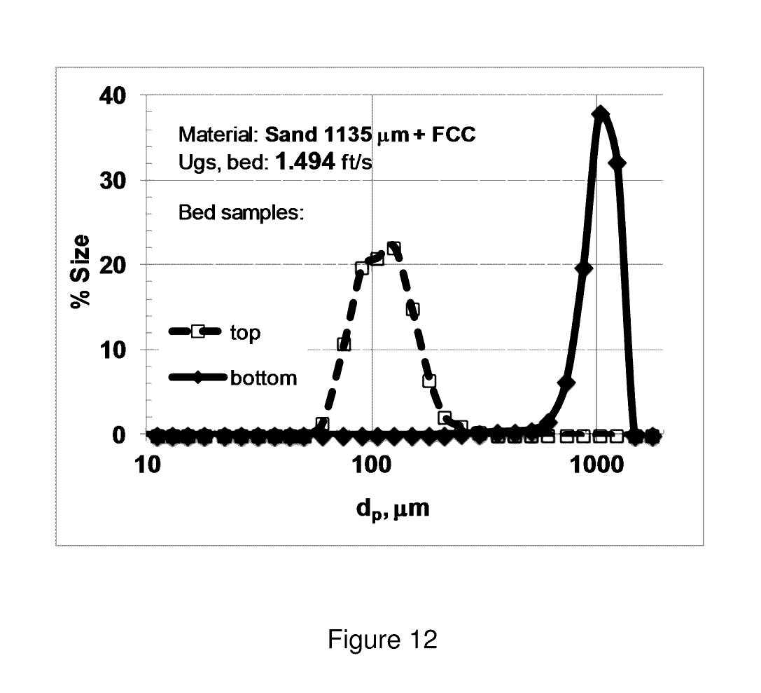

The second example uses the same FCC E-cat and a large sand particulate representing a surrogate hydrogen acceptor particulate having an average size of 1135 microns. The minimum fluidization velocity of this larger sand particulate with ambient condition air is about 2 ft/sec as compared to the minimum fluidization velocity of the FCC E-cat particulate of 0.008 ft/sec. As such, the ratio of the minimum fluidization velocity of the larger sand particulate (i.e., 2 ft/sec) to the minimum fluidization velocity of the FCC E-cat particulate (i.e., 0.008 ft/sec) is 250. In accordance with the disclosed subject matter, 250 is more than 5 and as such, the two particulates may be separated. However, in contrast to the presently disclosed subject matter, this ratio of 250 is not less than 200, and as such, the two particulates may not be well-mixed. FIG. 11 shows the pressure differential measurement in the upper and lower sections of the test bed at different superficial velocities. The measurements indicate that the two particulates appear to be separated at 1.494 ft/sec. Turning to FIG. 12, as shown, the measured particle size distribution depicted by open-square line markers was taken at the location of the top layer and the measured particle size distribution depicted by solid-diamond shaped line markers was taken at the location of the bottom layer of the bed. In FIG. 12, the additional measurements of direct samplings from the upper and lower sections of the bed demonstrate that the two particulates are indeed separated at a superficial velocity of 1.494 ft/sec. However, the mixing velocity of 2.686 ft/sec approaches the entrainment velocity of 4 ft/sec at which a portion of the FCC E-cat will no longer stay within the reaction zone.

Returning to FIG. 11, at a mixing velocity of 2.686 ft/sec, the two pressure differential measurements suggest that the two particulates appear to well-mixed; however, in reality, a portion of the FCC E-cat no longer stays within the reaction zone and cannot be considered to be well-mixed within the reaction zone. This second example demonstrates the case that when the minimum fluidization velocities of the two particulates are too dissimilar, the two particulates become difficult to stay mixed in the reaction zone.

The aforementioned advantages of the process of the disclosed subject matter provide for an efficient removal of hydrogen from the reaction zone of methane-containing gas aromatization reactor operating in fluidized bed mode and for shifting the reaction equilibrium towards higher methane-containing gas stream conversion and benzene yields per pass. Furthermore, according to the process of the disclosed subject matter, successful separation of the aromatization catalyst particulate from the hydrogen acceptor particulate may be achieved allowing for each particulate to be regenerated separately and subsequently returned to the aromatization reactor for further processing. Therefore, the disclosed subject matter has the potential to allow for the commercialization of an economically attractive direct, non-oxidative methane-containing gas stream aromatization process.

We claim:

1. A process for the aromatization of a methane-containing gas stream comprising:
contacting the methane-containing gas stream in a reaction zone comprising an aromatization catalyst particulate and a hydrogen acceptor particulate under methane-containing gas aromatization reaction conditions to produce reaction products comprising aromatics and gaseous hydrogen, wherein at least a portion of the gaseous hydrogen produced is bound by the hydrogen acceptor particulate in the reaction zone and removed from the reaction products in the reaction zone, and
separating the hydrogen acceptor particulate from the aromatization catalyst particulate in a separation zone under separation conditions.

2. The process of claim 1, wherein the aromatization catalyst particulate has a first set of physical properties comprising a first minimum fluidization velocity, and wherein the hydrogen acceptor particulate has a second set of physical properties comprising a second minimum fluidization velocity, and wherein the first minimum fluidization velocity is different from the second minimum fluidization velocity.

3. The process of claim 2, wherein the ratio of the second minimum fluidization velocity to the first minimum fluidization velocity is less than 200.

4. The process of claim 2, wherein the ratio of the second minimum fluidization velocity to the first minimum fluidization velocity is more than 15.

5. The process of claim 2, wherein the ratio of the first minimum fluidization velocity to the second minimum fluidization velocity is less than 200.

6. The process of claim 2, wherein the ratio of the first minimum fluidization velocity to the second minimum fluidization velocity is more than 15.

7. The process of claim 2, wherein the second minimum fluidization velocity is greater than the first minimum fluidization velocity, and wherein the aromatization reaction conditions comprise a superficial velocity that is greater than 1.5 times the second minimum fluidization velocity.

8. The process of claim 2, wherein the second minimum fluidization velocity is greater than the first minimum fluidization velocity, and wherein the separation conditions comprise a superficial velocity that is less than 1.5 times the second minimum fluidization velocity.

9. The process of claim 2, wherein the first minimum fluidization velocity is greater than the second minimum fluidization velocity, and wherein the aromatization conditions comprise a superficial velocity that is greater than 1.5 times the first minimum fluidization velocity.

10. The process of claim 2, wherein the first minimum fluidization velocity is greater than the second minimum fluidization velocity, and wherein the separation conditions comprise a superficial velocity that is less than 1.5 times the first minimum fluidization velocity.

11. The process of claim 1, wherein the separation conditions comprise a particulate residence time of more than 10 seconds.

12. The process of claim 1, wherein the separation zone is located in a separation vessel.

13. The process of claim 1, wherein the aromatization catalyst comprises a zeolite selected from the group consisting of ZSM-5, ZSM-22, ZSM-8, ZSM-11, ZSM-12 or ZSM-35.

14. The process of claim 1, wherein the aromatization catalyst comprises a metal selected from the group consisting of vanadium, chromium, manganese, zinc, iron, cobalt, nickel, copper, gallium, germanium, niobium, molybdenum, ruthenium, rhodium, silver, tantalum, tungsten, rhenium, platinum and lead and mixtures thereof.

15. The process of claim 1, wherein the aromatization catalyst particulate comprises a plurality of particles, each particle having a particle size in the range of 1 to 200 microns.

16. The process of claim 1, wherein the hydrogen acceptor comprises a metal or metals that are capable of selectively binding hydrogen under the methane-containing gas aromatization conditions in the reaction zone.

17. The process of claim 1, wherein the hydrogen acceptor comprises a metal selected from the group consisting of Ti, Zr, V, Nb, Hf, Co, Mg, La, Pd, Ni, Fe, Cu, Ag, Cr, Th and other transition metals and compounds or mixtures thereof.

18. The process of claim 1, wherein the hydrogen acceptor particulate comprises a plurality of particles, each particle having a particle size in the range of 100-2000 microns.

19. The process of claim 1, further comprising continuously regenerating the aromatization catalyst to remove coke formed during the reaction under first regeneration conditions in a first regeneration vessel.

20. The process of claim 1, further comprising continuously regenerating the hydrogen acceptor by releasing the hydrogen under second regeneration conditions in a second regeneration vessel.
